(19) United States
(12) Patent Application Publication
(10) Pub. No.: US 2018/0207450 A1
(43) Pub. Date: Jul. 26, 2018

(54) DEVICES AND METHODS FOR SELECTIVELY ACTIVATING AFFERENT NERVE FIBERS

(71) Applicant: Aucta Technologies Inc., Minneapolis, MN (US)

(72) Inventors: Hector Daniel Romo Sanchez, West Saint Paul, MN (US); Daniel Withers Gulick, Phoenix, AZ (US)

(21) Appl. No.: 15/926,094

(22) Filed: Mar. 20, 2018

Related U.S. Application Data
(63) Continuation of application No. PCT/US2017/055067, filed on Oct. 4, 2017.
(60) Provisional application No. 62/404,196, filed on Oct. 4, 2016.

(51) Int. Cl. A61N 7/02; A61N 1/36

(52) U.S. Cl. CPC A61N 7/02; A61N 2007/0026; A61N 1/3603

(57) ABSTRACT

Implementations described herein include a device including a stimulation transducer operably coupled to a stimulation controller. The stimulation transducer focally delivers energy to a target tissue at a selected depth below the surface of the skin of a subject and the stimulation controller generates a signal indicating a selected pulse intensity, a selected pulse duration, and a selected treatment session duration sufficient to heat the target tissue to a temperature from 40 to 45 degrees Celsius via application of energy by the stimulation transducer. When the stimulation transducer receives the signal from the stimulation controller, the stimulation transducer focally delivers energy having the selected pulse intensity, the selected pulse duration, and the selected treatment session duration to heat the target tissue to selectively and reversibly activate afferent nerve fibers having thermo-sensitive ion channels to send sensory information to the central nervous system without causing any permanent changes to the target tissue.

### RELATED APPLICATIONS

This application is a continuation of International Application No. PCT/US2017/055067, filed Oct. 4, 2017, and claims the benefit of priority to U.S. Provisional Application Ser. No. 62/404,196, filed Oct. 4, 2016, all of which are incorporated hereby by reference in their entirety.

### FIELD

The present disclosure relates to devices for selectively activating afferent nerve fibers and associated methods.

### BACKGROUND

This section provides background information related to the present disclosure, which is not necessarily prior art.

Current neuromodulation technology includes the use of implantable and non-invasive devices that can stimulate or inhibit neurons and nerve fibers in the central or peripheral nervous system. Available neuromodulation devices utilize energy in the form of electrical current, pulsed magnetic fields, ultrasound, infrared, and optogenetics to modulate nervous tissue. Neuromodulation devices utilizing energy to heat or induce hyperthermia in nerves effect permanent or semi-permanent ablation or blocking of the target nerves. Such devices and stimulation techniques are non-selective in that they stimulate all fiber types to target the largest nerve fibers and stimulate both afferent and efferent directions.

By far, the most utilized and studied of the neuromodulation techniques is stimulation by electrical current. Electrical stimulation of a nerve preferentially stimulates large diameter fibers (e.g., Aα and Aβ fibers), which are generally myelinated and fast conducting fibers, and activates both afferent and efferent fibers. Electrical stimulation can only stimulate both small and large fibers by using pulses having relatively high intensities and/or long durations, such as by using about 40 times more voltage than that required to activate the large fibers alone. These high intensities and long durations, alone or in combination, excessively activate large diameter nerve fibers and can cause pain in the subject if intended to additionally activate small diameter/slow conducting fibers.

High- to medium-intensity (>500 mW/cm²), high frequency (>1 MHz) ultrasound has also been utilized to effect hyperthermic neuromodulation. This neuromodulation has long-lasting effects on the nerve, affecting conduction for more than 1 hour after the end of the ultrasound application. This includes both irreversible ablation of nerve fibers and reversible long-lasting disruption of nerve fibers. This form of neuromodulation blocks the conduction of action potentials.

Low-intensity (<500 mW/cm²), low frequency (<0.9 MHz) ultrasound has solely been used in the realm of neuromodulation to induce pressure fluctuations. These pressure fluctuations can stimulate neurons by a mechanism that is currently not well understood. It is known that this form of low-intensity low-frequency neuromodulation is not thermal, that is, the neuron stimulation is not directly due to tissue heating. In fact, such methods seek to minimize any heating as it is regarded as a parasitic side effect of the low-intensity, low frequency ultrasound.

### SUMMARY

The present inventors have recognized, among other things, that a problem to be solved can include reversibly and, optionally, selectively activating afferent nerve fibers having thermo-sensitive ion channels to send sensory information to the central nervous system without causing any permanent and, optionally, long-lasting changes to the target tissue. The present subject matter can help provide a solution to this problem, such as by providing a device comprising a stimulation transducer operably coupled to a stimulation controller. The stimulation transducer can focally deliver energy to a target tissue at a selected depth below the surface of the skin of a subject. The stimulation controller can generate a signal indicating a selected pulse intensity, a selected pulse duration, and a selected treatment session duration sufficient to heat the target tissue to a temperature of 40 to 45 degrees Celsius via application of energy by the stimulation transducer. When the stimulation transducer receives the signal from the stimulation controller, the stimulation transducer can focally deliver energy having the selected pulse intensity, the selected pulse duration, and the selected treatment session duration to heat the target tissue to selectively and reversibly activate the afferent nerve fibers with thermo-sensitive ion channels to send sensory information to the central nervous system without causing any long-lasting or permanent changes to the target tissue.

The present subject matter can help provide another solution to this problem, such as by providing a method that can selectively and reversibly activate afferent nerve fibers having thermo-sensitive ion channels to send sensory information to the central nervous system without causing any long-lasting or permanent changes to the target tissue. Such methods can comprise steps such as generating a signal indicating a selected pulse intensity, a selected pulse duration, and a selected treatment session duration sufficient to heat a target tissue to a temperature of 40 to 45 degrees C. via a stimulation controller, the target tissue of a subject comprising a target nerve comprising afferent nerve fibers with thermo-sensitive ion channels; transmitting the signal to a stimulation transducer; generating focalized energy with the selected pulse intensity, the selected pulse duration, and the selected treatment session duration; applying the focalized energy to the target tissue at a selected depth below the surface of the skin of a subject; heating the target tissue to selectively and reversibly activate the afferent nerve fibers with thermo-sensitive ion channels to send selectively sensory information to a central nervous system of the subject without causing any long-lasting or permanent changes to the target tissue.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a perspective view of one exemplary embodiment of a device according to the present disclosure in use.

FIG. 2 illustrates a schematic view of one exemplary embodiment of a device according to the present disclosure.

FIG. 3 illustrates a schematic showing the basic components of the embodiments of at least FIGS. 1 and 2.

FIG. 4 is a flow chart illustrating one exemplary method according to the present disclosure.

FIG. 5 illustrates the effects of focal increase in temperature to a target temperature according to the present disclosure.

FIG. 6 is a graph illustrating reduction of the glycemia spike after an oral glucose tolerance test upon vagus nerve thermo-sensitive fiber stimulation.

FIG. 7 is a graph illustrating that vagus nerve thermo-sensitive fiber stimulation reduces glycemia to a lesser extent than when applied without a glucose challenge.

FIG. 8 is a graph illustrating that vagus nerve thermo-sensitive fiber stimulation is undistinguishable from sham stimulation when measuring variation in heart rate.

FIG. 9 is a graph illustrating that vagus nerve thermo-sensitive fiber stimulation is able to reduce TNF-alpha whole blood levels when compared with baseline.

### DETAILED DESCRIPTION

The present subject matter provides for devices and methods that can reversibly activate small diameter nerve fibers such as, for example and without limitation, afferent nerve fibers having thermo-sensitive ion channels and the like, to send sensory information to the central nervous system by using heat to stimulate a target tissue and without causing any permanent changes to the target tissue. Small diameter nerve fibers can include Aδ and C fibers. Small diameter nerve fibers carry somatic sensations, transfer information between organs and the brain, are active in both the sympathetic and parasympathetic nervous system, and the like. Many vital physiological functions are modulated by the activity of small diameter nerve fibers such as, for example and without limitation, heart rate, breathing, digestion, and the like. The selective modulation of small diameter nerve fibers, such as afferent nerve fibers having thermo-sensitive ion channels and the like, can enable manipulation of physiological functions for the treatment of disease or the enhancement of physiological processes as further described herein.

In one example, the present subject matter comprises a device comprising a stimulation transducer operably coupled to a stimulation controller. The stimulation transducer can focally deliver energy to a target tissue. The stimulation controller can generate a signal indicating a selected pulse intensity, a selected pulse duration, and a selected treatment session duration sufficient to heat the target tissue to a temperature of 40 to 45 degrees Celsius via application of energy by the stimulation transducer. When the stimulation transducer receives the signal from the stimulation controller, the stimulation transducer can focally deliver energy having the selected signal characteristics to heat the target tissue to reversibly and, optionally, selectively activate afferent nerve fibers with thermo-sensitive ion channels to send sensory information to the central nervous system without causing any permanent changes to the target tissue. It is further contemplated that the device does not cause any long-lasting changes to the target tissue, such long-lasting changes including structural lesions, pathophysiological alterations, and the like. Such devices and methods can use medium intensity focused energy, such as, but not limited to, ultrasound energy, radiofrequency (RF) energy, and direct conductive heating, and the like to stimulate afferent nerve fibers with thermo-sensitive ion channels in order to invoke somatic or neural effects to manipulate physiological functions to treat disease or to enhance physiological processes as explained in further detail below.

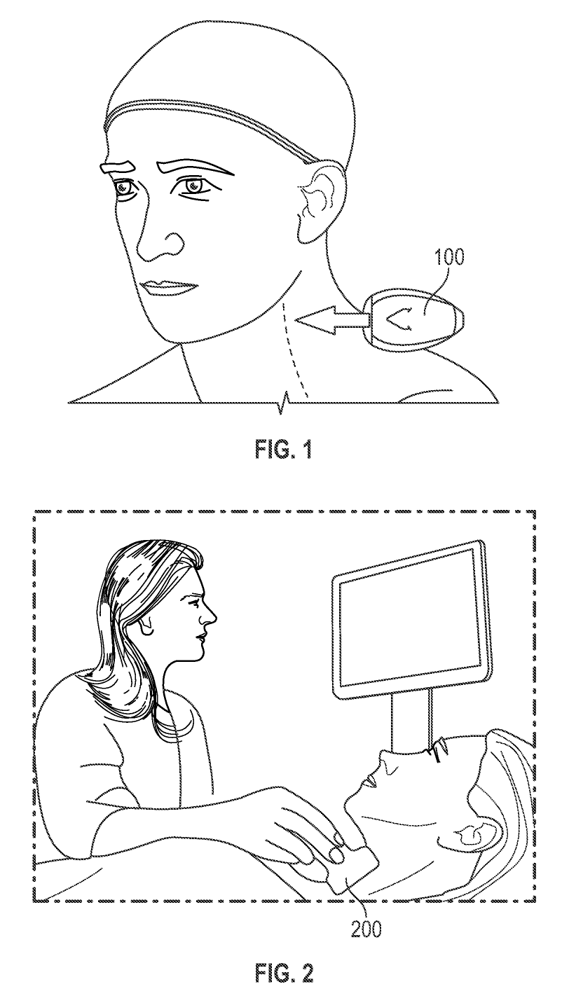

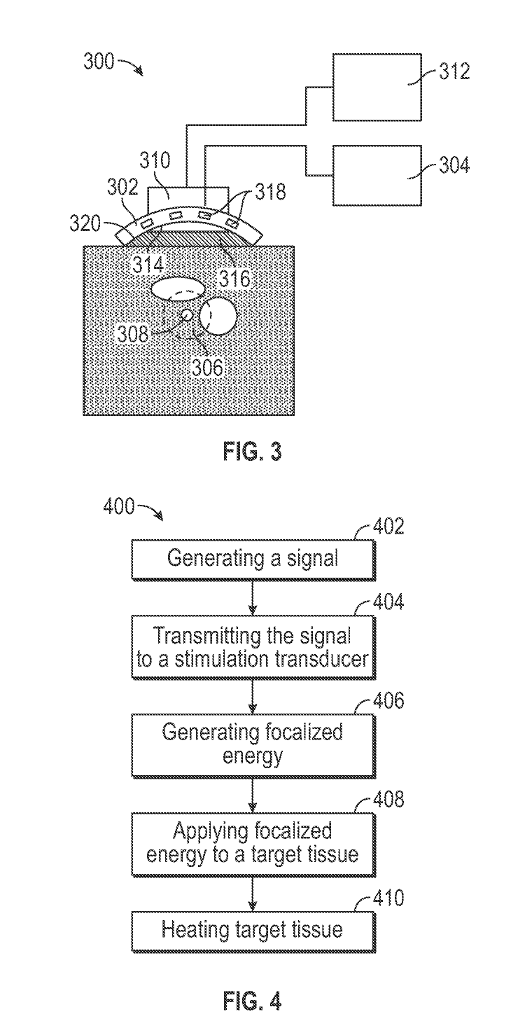

In one exemplary embodiment illustrated in FIG. 1, the device 100 can comprise a non-invasive device that can be handheld, can have a rechargeable power source, and can accommodate self-stimulation by a user or a healthcare provider. In another exemplary embodiment illustrated in FIG. 2, the device 200 can comprise a non-invasive device that can be stationary and can receive power from a wall socket. FIG. 3 illustrates a schematic view of the main components of the devices of at least FIGS. 1 and 2. The device 300 can comprise a stimulation transducer 302 operably coupled to a stimulation controller 304. The stimulation transducer 302 can focally deliver energy to a target tissue 306 at a selected depth below the surface of the skin of a subject. In an example, the selected depth below the surface of the skin of the subject excludes the surface of the skin of the subject. In an example, energy can be focally delivered to target tissue at the selected depth, with non-target tissue above and below the target tissue receiving less stimulation effect (e.g., heating) than the target tissue. The target tissue can comprise a target nerve 308 comprising afferent nerve fibers with thermo-sensitive ion channels. The afferent nerve fibers with thermo-sensitive ion channels can comprise Aδ or C fibers. The thermo-sensitive ion channels can comprise one or more of TRPV1, TRPV2, TRPV3, TRPM2, and TRPM3. The stimulation controller 304 can generate a signal indicating a selected pulse intensity, a selected pulse duration, and a selected treatment session duration sufficient to heat the target tissue 306 to a temperature of 40 to 45 degrees Celsius (e.g., 43 degrees Celsius), via application of energy by the stimulation transducer 302. When the stimulation transducer 302 receives the signal from the stimulation controller 304, the stimulation transducer 302 can focally deliver energy having the selected pulse intensity, the selected pulse duration, and the selected treatment session duration to heat the target tissue 306 to selectively and reversibly activate the afferent nerve fibers with thermo-sensitive ion channels to send sensory information to the central nervous system without causing any permanent or, optionally, long-lasting changes to the target tissue. The stimulation transducer 302 can incrementally heat the target tissue 306 to desensitize the subject to temperature increases, thereby avoiding intolerable pain. As thermo-sensitive ion channels are present in the receptive ending and also along the length of the axon, it is further contemplated that stimulation can be effected along several locations in the orthogonal trajectory of the nerve. In one example, stimulating multiple locations along a nerve can allow one location to cool-down while another location along the same nerve can be stimulated to transmit nerve impulses to the central nervous system.

In one exemplary embodiment, the stimulation transducer 302 can further comprise an ultrasound transducer and the stimulation controller 304 can further comprise an ultrasound controller. A distal end of the stimulation transducer 302 include a transduction medium interface 314, such as for receiving an ultrasound transmission medium 316 such as gel or the like. The stimulation transducer 302 can generate ultrasound frequencies from 2 to 10 MHZ (e.g., 3 to 8 MHz, 4 to 6 MHz, etc.). The stimulation transducer 302 can comprise at least one stimulation element 318. In a further example, the stimulation transducer can comprise an array of stimulation elements 318. The array of stimulation elements 318 can comprise an array of 2 to 2000 stimulation elements (e.g., 2 to 100 stimulation elements, etc.).

The stimulation transducer 302 can comprise a contact area with skin from 1 cm² to 30 cm² (e.g., 2 cm² to 20 cm², 3 cm² to 10 cm², etc.). The stimulation transducer 302 can deliver energy focally across an adjustable depth range, the selected depth being in the adjustable depth range. The adjustable depth range can be from 0.1 cm to 30 cm (e.g., 0.3 cm to 20 cm, 0.5 to 10 cm, etc.). The target tissue 306 can have an area of 0.1 mm² to 20 cm² (1 mm² to 10 cm², 5 mm² to 5 cm², etc.). Such ranges of contact areas have sufficient cross-sectional dimensions to reliably focally deliver energy to the target tissue 306 and the selected depth below the surface of the skin of the subject and across the area of the target tissue 306 to heat the target tissue 306 to the selected temperature of 40 degrees C. to 45 degrees C. while avoiding hyperthermic effects that can include intolerable pain to the subject and any long-lasting or permanent changes to the target tissue 306.

In some embodiments, the selected pulse intensity can be from 10 to 200 W/cm² (e.g., 20 to 100 W/cm², 30 to 60 W/cm², etc.). Additionally or alternatively, the selected pulse duration can be from 1 second to 10 minutes (e.g.,3 seconds to 5 minutes, 3 seconds to 30 seconds, etc.). Additionally or alternatively, the selected treatment session duration can be from 1 second to 24 hours (e.g., 1 minute to 30 minutes, etc.). In light of the present disclosure, a skilled artisan will appreciate that the signal characteristics of intensity, pulse duration, and session duration are interrelated. The selection of these signal characteristics is governed by the desired level of tissue heating. For example, given a particular pulse duration and a particular intensity to achieve a particular tissue temperature, a shorter pulse duration and a higher intensity can also be utilized to achieve the particular tissue temperature. Further, certain ranges are disclosed, providing a range of power efficiency, efficacy, and effect to target and non-target tissue. In certain examples, the ranges may increase or decrease with evolving technology. For example, a stimulation transducers evolve, the range of contact areas disclosed herein may increase or decrease, while still providing the desired effect. Other ranges will evolve similarly, such as through material or technology advancements, and are included herein.

In some embodiments, the device 300 can comprise a protective element 320 to mitigate any rise in temperature at the skin of the subject due to device 300 heating. Running high power through ultrasound transducers can increase their temperature considerably and may create discomfort or pain in the skin of a subject. The protective element 320 can comprise an insulator or a convective cooling element. The convective cooling element can comprise a fan, a circulated coolant, or the like.

In some embodiments, the device 300 can further comprise an imaging system comprising an imaging transducer 310 and an imaging controller 312 operably coupled to the imaging transducer 310. The imaging system can comprise a Doppler ultrasound system, a 2-D ultrasound imaging system, or the like.

Doppler ultrasound sensing of arterial and/or venous blood vessels can be used as a proxy to infer the location of the target nerve. In one embodiment, the vagus nerve in the neck can be located between the carotid artery and the jugular vein by Doppler ultrasound because of their strong pulsating signal of the carotid artery. The carotid artery landmark can be used to provide a feedback signal for an automated or user-controlled positioning system to aim the focused ultrasound beam at the target nerve 308 with reference to the location of a blood vessel or vessels. In one example, a single-beam Doppler ultrasound system can be incorporated into the device 300. The Doppler beam can be swept by the user back and forth in one or several axes until the angle that gives the greatest pulsatile blood flow signal is found, at which point the device 300 may cue the user that the device 300 is in a correct position. The Doppler output signal can be processed into an audio or visual feedback signal to the user. Within the system, the Doppler beam and the stimulation beam can have an angle between them such that aiming the Doppler beam directly at the carotid artery aims the power ultrasound beam directly at the vagus nerve. This angle can be adjustable, and a medical professional may set the angle by using an image (e.g., ultrasound MRI, or CT) of the neck to determine the correct angle between the Doppler beam and the stimulation beam generated by the stimulation transducer 302. Such a device can be a partially closed-loop aiming device in that it provides a feedback signal to enable the user to find and maintain proper aim of the stimulation beam in reference to the target nerve.

In another embodiment, the imaging system can comprise a 2-D ultrasound imaging system that can be incorporated into the device 300 to locate the vagus nerve by reference to the nearby landmarks, such as the carotid artery, jugular vein, and the like. Such a device can be a closed-loop aiming system, with the device 300 automatically tracking the position of the target nerve 308 and adjusting the aim of the power beam to track the target nerve 308.

FIG. 4 is a flow chart illustrating an exemplary method according to the present invention. Such a method 400 can begin with generating a signal 402, the signal indicating a selected pulse intensity, a selected pulse duration, and a selected treatment session duration sufficient to heat a target tissue to a temperature of 40 to 45 degrees C. via a stimulation controller. The target tissue of a subject can comprise a target nerve comprising afferent nerve fibers with thermo-sensitive ion channels. The target tissue can comprise a target nerve comprising afferent nerve fibers with thermo-sensitive ion channels. The afferent nerve fibers with thermo-sensitive ion channels can comprise Aδ or C fibers. The thermo-sensitive ion channels can comprise one or more of TRPV1, TRPV2, TRPV3, TRPM2, and TRPM3. Next, the method 400 can include transmitting the signal to a stimulation transducer 404 and generating focalized energy with the selected pulse intensity, the selected pulse duration, and the selected treatment session duration 406 via the stimulation transducer. Next, the method 400 can include applying the focalized energy to the target tissue at a selected depth below the surface of the skin of a subject 408 and heating the target tissue 410 to selectively and reversibly activate the afferent nerve fibers with thermo-sensitive ion channels to send selectively sensory information to a central nervous system of the subject without causing any long-lasting or permanent changes to the target tissue. Heating the target tissue 410 can further comprise incrementally heating the target tissue to the temperature. Optionally, the method 400 can further comprise selecting the target tissue based on the target nerve.

In other embodiments, the method 400 can further comprise imaging the subject to locate the target nerve. Imaging the target nerve can comprise using an imaging system (e.g., an ultrasound, an MRI, or a CT) can be used in an initial calibration session to collect information to determine baseline information such as target tissue location and characteristics that can be used in subsequent treatment according to the above methods in order to aim the focalized energy at one or several points along the target nerve. In one example, such baseline information can be used to build a bracket to hold at least a portion of the device at a constant position relative to nearby anatomical landmarks such as the neck, the jawbone, the collarbone, artificial landmarks (e.g., tattoos or implanted magnets), and the like.

Additionally or alternatively, the method 400 can include using a reference guide to facilitate correct positioning of the device. In one example, the reference guide can cooperate with anatomical landmarks on the body, tattoos, implanted subcutaneous magnets, and the like in order to ensure correct alignment of the device. In another example, the reference guide can generate auditory cues as feedback in order to tell an operator or the subject when the stimulation unit is correctly positioned and ready for use.

In additional embodiments, the method 400 can include calibrating the signal. The signal can be calibrated based on the threshold of activation of pain receptors or a referred sensation reported by the subject. Additionally or alternatively, the signal can be calibrated based on a depth of the nerve from the skin surface and local tissue characteristics to ensure the selected depth, the selected pulse intensity, the selected pulse duration, and the selected treatment session duration are sufficient to heat the target nerve to the temperature of 40 degrees Celsius to 45 degrees Celsius.

In additional embodiments, the method 500 can employ ultrasound. Here, the stimulation transducer 302 can comprise an ultrasound transducer and the stimulation controller 304 can comprise an ultrasound controller. Generating the focalized energy can further comprise generating an ultrasound frequency from 2 to 10 MHz. The selected pulse intensity of the signal can be from 10 to 200 W/cm² (e.g., 20 to 100 W/cm², 30 to 60 W/cm², etc.). The selected pulse duration of the signal can be from 1 second to 10 minutes (e.g., 3 seconds to 5 minutes, 3 seconds to 30 seconds, etc.). The selected treatment session duration of the signal can be from 1 second to 24 hours (e.g., 1 minute to 30 minutes, etc.).

In additional embodiments, the step of applying the focalized energy to the target tissue 306 at the selected depth can further comprise applying the stimulation transducer 302 to a contact area of skin from 1 cm² to 30 cm² (e.g., 2 cm² to 20 cm², 3 cm² to 10 cm², etc.).

In additional embodiments, the method 400 can further comprise determining the selected depth from an adjustable depth range of the stimulation transducer. The adjustable depth range can be from 0.1 cm to 30 cm (e.g., 0.3 cm to 20 cm, 0.5 cm to 10 cm, etc.). The target tissue can have an area of 0.1 mm² to 20 cm² (e.g., 1 mm² to 10 cm², 5 mm² to 5 cm², etc.).

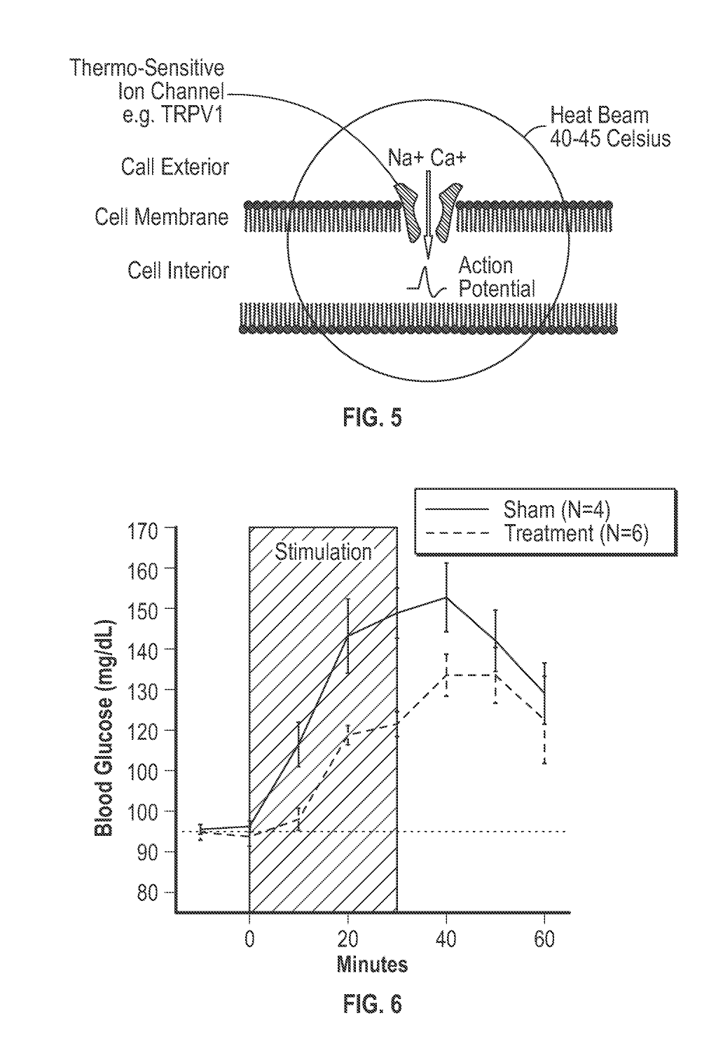

FIG. 5 illustrates the effects of focal increase in temperature to a target temperature according to the present disclosure. The focal increase of temperature on the target tissue can create action potentials by opening thermo-sensitive ion channels in the membranes of nerve cells. These channels can open at temperatures from 40 to 45 degrees Celsius, thereby allowing sodium and/or calcium ions into the cell, depolarizing the membranes, and generating an action potential. Thermo-sensitive ion channels that can open at these temperatures include TRPV1, TRPV2, TRPV2, TRPM2, TRPM3, and the like. Such channels are typically only expressed in small diameter, slow conducting, unmyelinated or lightly-myelinated, afferent nerve fibers. Further, the firing rate of thermo-sensitive nerve fibers can increase with increased temperature, enabling a dose-dependent control of the stimulation via control of the magnitude of the temperature rise. While focal heating can affect the entire nerve bundle of the target tissue, only nerve fibers having thermo-sensitive ion channels will be activated. The depolarization of the axon is independent of structural lesions, pathophysiological alternations, or the like typically associated with hyperthermia-based treatment modalities such as ablation. Accordingly, in light of the present disclosure, a skilled artisan will appreciate that the selective activation of small sensory nerve fibers with thermo-sensitive ion channels enables entirely new therapies based on autonomic nerve stimulation. In one non-limiting example, selective stimulation of the vagus nerve can send signals to the central nervous system about the general state of organs such as, but not limited to, the pancreas, the liver, the spleen, and the like. The vagus nerve comprises approximately 80% afferent fibers and many of these fibers also comprise thermo-sensitive ion channels.

FIG. 6 illustrates reduction of blood glucose levels with thermo-sensitive fiber stimulation applied to the cervical trunk of the left vagus nerve according to the present disclosure. A healthy subject administered a glucose test and was subjected to treatment via the devices and methods described herein using ultrasound-induced heating applied for 3 seconds each minute for a total of 30 minutes and shows significantly reduced blood glucose levels during an oral glucose tolerance test. The active stimulations (focused on the vagus nerve) reduced the glucose spike (relative to baseline) in capillary blood by 57% when compared with sham stimulations (focused on a nearby muscle).

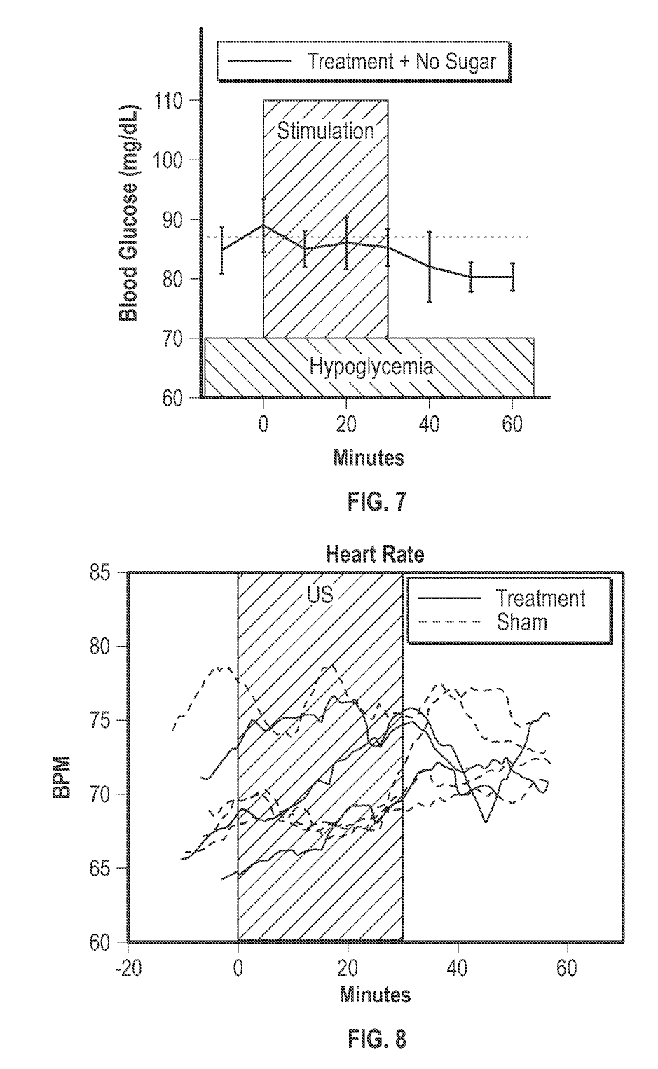

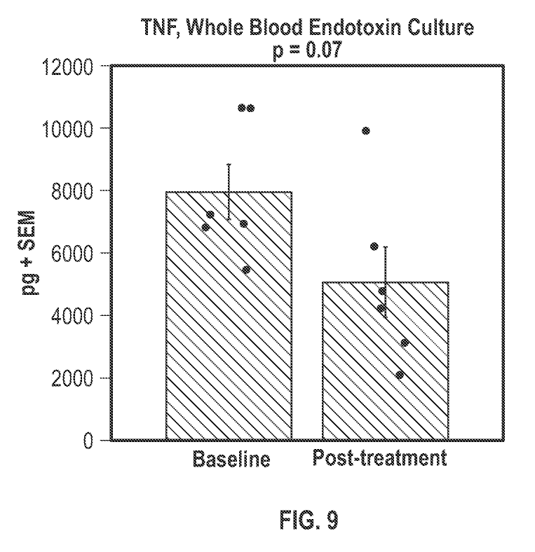

It is further contemplated that the present devices and methods can increase the frequency of afferent signaling to the central nervous system, causing the brain to perceive a physiological condition and increase the efferent activation of a tissue or organ by the central nervous system. For example, using the present devices and methods as described above with respect to at least FIG. 6 can increase the afferent signaling of the vagus nerve to the central nervous system, causing the central nervous system to perceive high glucose levels and therefore increase the efferent activation of the pancreas by the central nervous system. FIG. 7 illustrates how, in the absence of glucose ingestion, the vagus nerve thermal stimulation via the present devices and methods can reduce glycemia to a lesser extent than when applied during the glucose test (reduction with glucose: approx. 30 mg/dL, reduction without glucose: approx. 10 mg/dL), suggesting that the glycemia reduction may be mediated by a glucose-dependent insulin secretion in the pancreas. Furthermore, FIG. 8 illustrates how vagus nerve thermo-sensitive fiber stimulation via the present devices and methods can be indistinguishable from sham stimulation when measuring heart rate changes.

In another example, the present devices and methods can increase sensory nerve signaling to the central nervous system and, more particularly, the brain, during acute injury, which may help the body maintain improved sensory feedback, enabling an improved healing process. It is contemplated that the present devices and methods can be used to counteract decreased vagal nerve activity related to acute myocardial infarction and, thus, the increased mortality associated therewith. Increasing the vagus signaling from the site of injury via the present devices and methods may increase the responsiveness of the brain, altering the brain's output to trigger-improved management of traumatic injury, infection, and other forms of acute injury. The present devices and methods can stimulate thermo-sensitive ion channels in the vagus nerve which can reduce TNF-alpha release levels when compared with baseline in a healthy subject as illustrated in FIG. 9. TNF-alpha can be a necessary and sufficient biomarker of systemic inflammation and TNF-alpha release can be reduced by invoking the inflammatory reflex, a neural circuit in the vagus nerve. The inflammatory reflex comprises an afferent and efferent arc and both types of fibers run through the vagus nerve. Accordingly, thermo-sensitive ion channel stimulation via the present devices and methods, although only targeted to activate afferent fibers, can also initiate neural circuits with efferent arcs such as the inflammatory reflex.

Accordingly, the present devices and methods can selectively stimulate a narrower subset of fibers that can be sufficient to induce autonomic effects like glucose reduction without causing material variations in vital signs such as heart rate, blood pressure, and the like. As a result, the present devices and methods can allow for safer stimulation as well as allow for new therapies that were not previously possible due to side effects inherent to other non-selective stimulation methods.

The above is but one example of using the present devices and methods to generate sensory information to the central nervous system to enable significant therapeutic effects regulated through the central nervous system (rather than by direct stimulation of efferent fibers) and wherein the central nervous system acts like a buffer for the final neural output that the tissue or organ will receive. In one example, several hormonal and non-hormonal receptors are less present in the vagus nerve during chronic disease. For instance, obesity can reduce the expression of receptors for leptin in vagus nerve fibers. As well, the impulse conduction of the vagus nerve can weaken during the progression of obesity. Long-term repeated activation of sensory/afferent fibers by thermal stimulation via the present devices and methods may help maintain the nerve by enhancing the subject's vagal tone and thereby maintaining the neural pathway connecting the gut to the brain. Ultimately, it is contemplated that regular sensory stimulation via the present devices and methods may help maintain body homeostasis and prevent disease.

Additional examples of chronic diseases that can be treated with the present devices and methods include, but are not limited to, diabetes mellitus, obesity, metabolic syndrome, heart failure, other heart conditions, chronic obstructive pulmonary disease, hypertension, hepatitis, epilepsy, depression, schizophrenia, chronic traumatic encephalopathy, rheumatoid arthritis, post-traumatic stress disorder, anxiety disorders, obsessive compulsive disorder, other psychiatric disorders, Parkinson's disease, Crohn's disease, autoimmune diseases, chronic pain, overlapping pain conditions, chronic low back pain, fibromyalgia, chronic migraine, colitis, irritable bowel syndrome, endometriosis, cancer, spinal cord injuries, Alzheimer's disease, coma, vegetative states, addiction, asthma, and other pathologies associated with organs of the human or animal body. Additional examples of acute diseases that can be treated with the present devices and methods include, but are not limited to, traumatic brain injury, burns, concussions, ischemia-reperfusion injury, endotoxemia, seizures, stroke, acute myocardial infarction, stress-induced hyperglycemia, pancreatitis, acute lung injury, renal ischemic reperfusion, post-cardiac arrest, artery occlusion, acute kidney injury, hemorrhagic shock, hemorrhage, sepsis, cephalalgia, viral infections, bacterial infections, fungus infections, allergies, postoperative ileus, episodic anxiety crisis, bone fractures, sprains, strains, tendinitis, fasciitis, bursitis, asthma exacerbations, surgery, allergic reactions, and other pathologies associated with organs of the human or animal body.

A list of numbered examples according to the present subject matter follow:

Example 1 is a device, comprising: a stimulation transducer that focally delivers energy to a target tissue at a selected depth below the surface of the skin of a subject, the target tissue comprising a target nerve comprising afferent nerve fibers with thermo-sensitive ion channels; and a stimulation controller operably coupled to the stimulation transducer, wherein the stimulation controller generates a signal indicating a selected pulse intensity, a selected pulse duration, and a selected treatment session duration sufficient to heat the target tissue to a temperature of 40 to 45 degrees Celsius via application of energy by the stimulation transducer; wherein, when the stimulation transducer receives the signal from the stimulation controller, the stimulation transducer focally delivers energy having the selected pulse intensity, the selected pulse duration, and the selected treatment session duration to heat the target tissue to reversibly activate the afferent nerve fibers with thermo-sensitive ion channels to send sensory information to the central nervous system without causing any permanent changes to the target tissue.

In Example 2, the subject matter of Example 1 optionally includes wherein the stimulation transducer comprises an ultrasound transducer and wherein the stimulation controller comprises an ultrasound controller.

In Example 3, the subject matter of Example 2 optionally includes wherein a distal end of the stimulation transducer includes a transduction medium interface.

In Example 4, the subject matter of any one or more of Examples 2-3 optionally include wherein the stimulation transducer generates ultrasound frequencies from 2 to 10 MHz.

In Example 5, the subject matter of Example 4 optionally includes wherein the stimulation transducer generates ultrasound frequencies from 3 to 8 MHz.

In Example 6, the subject matter of Example 5 optionally includes wherein the stimulation transducer generates ultrasound frequencies from 4 to 6 MHz.

In Example 7, the subject matter of any one or more of Examples 2-6 optionally includes wherein the selected pulse intensity is from 10 to 200 W/cm².

In Example 8, the subject matter of Example 7 optionally includes wherein the selected pulse intensity is from 20 to 100 W/cm².

In Example 9, the subject matter of Example 8 optionally includes wherein the selected pulse intensity is from 30 to 60 W/cm².

In Example 10, the subject matter of any one or more of Examples 2-9 optionally include wherein the selected pulse duration is from 1 second to 10 minutes.

In Example 11, the subject matter of Example 10 optionally includes wherein the selected pulse duration is from 3 seconds to 5 minutes.

In Example 12, the subject matter of Example 11 optionally includes wherein the selected pulse duration is from 3 seconds to 30 seconds.

In Example 13, the subject matter of any one or more of Examples 2-12 optionally include wherein the selected treatment session duration is from 1 second to 24 hours.

In Example 14, the subject matter of Example 13 optionally includes wherein the selected treatment session duration is from 1 minute to 30 minutes.

In Example 15, the subject matter of any one or more of Examples 1-14 optionally include wherein the stimulation transducer comprises a contact area with skin of 2 cm² to 20 cm².

In Example 16, the subject matter of Example 15 optionally includes wherein the stimulation transducer comprises a contact area with skin of 2 cm² to 20 cm².

In Example 17, the subject matter of Example 16 optionally includes wherein the stimulation transducer comprises a contact area with skin of 3 cm² to 10 cm².

In Example 18, the subject matter of any one of Examples 1-17 optionally includes wherein the stimulation transducer comprises at least one stimulation element.

In Example 19, the subject matter of Example 18 optionally includes wherein the stimulation transducer comprises an array of 2 to 2000 stimulation elements.

In Example 20, the subject matter of Example 19 optionally includes wherein the stimulation transducer comprises an array of 2 to 100 stimulation elements.

In Example 21, the subject matter of any one of Examples 1-20 optionally include wherein the target tissue has an area of 0.1 mm² to 20 cm².

In Example 22, the subject matter of Example 21 optionally includes wherein the target tissue has an area of 1 mm² to 10 cm².

In Example 23, the subject matter of Example 21 optionally includes wherein the target tissue has an area of 5 mm² to 5 cm².

In Example 24, the subject matter of any one or more of Examples 1-23 optionally include wherein the stimulation transducer delivers energy focally across an adjustable depth range, the selected depth being selected from the adjustable depth range.

In Example 25, the subject matter of Example 24 optionally includes wherein the adjustable depth range is from 0.1 cm to 30 cm.

In Example 26, the subject matter of Example 25 optionally includes wherein the adjustable depth range is from 0.3 cm to 20 cm.

In Example 27, the subject matter of Example 26 optionally includes wherein the adjustable depth range is from 0.5 cm to 10 cm.

In Example 28, the subject matter of any one or more of Examples 1-27 optionally include an imaging transducer and an imaging controller operably coupled to the imaging transducer.

In Example 29, the subject matter of any one or more of Examples 1-28 optionally include wherein the afferent nerve fibers with thermo-sensitive ion channels comprise Aδ or C fibers.

In Example 30, the subject matter of any one or more of Examples 1-29 optionally include wherein the afferent nerve fibers with thermo-sensitive ion channels comprise Aδ or C fibers.

In Example 31, the subject matter of any one or more of Examples 1-30 optionally include wherein the stimulation transducer selectively activates the afferent nerve fibers with thermo-sensitive ion channels.

In Example 32, the subject matter of any one or more of Examples 1-31 optionally include wherein the stimulation transducer does not cause any long-lasting changes to the target tissue.

Example 33 is a method, comprising: generating a signal indicating a selected pulse intensity, a selected pulse duration, and a selected treatment session duration sufficient to heat a target tissue to a temperature of 40 to 45 degrees C. via a stimulation controller, the target tissue of a subject comprising a target nerve comprising afferent nerve fibers with thermo-sensitive ion channels; transmitting the signal to a stimulation transducer; generating focalized energy with the selected pulse intensity, the selected pulse duration, and the selected treatment session duration; applying the focalized energy to the target tissue at a selected depth below the surface of the skin of a subject; heating the target tissue to selectively and reversibly activate the afferent nerve fibers with thermo-sensitive ion channels to send selectively sensory information to a central nervous system of the subject without causing any long-lasting or permanent changes to the target tissue.

In Example 34, the subject matter of Example 33 optionally includes selecting the target tissue based on the target nerve.

In Example 35, the subject matter of Example 34 optionally includes imaging the subject to locate the target nerve.

In Example 36, the subject matter of Example 35 optionally includes calibrating the signal based on a depth of the nerve from the skin surface and local tissue characteristics to ensure the selected depth, the selected pulse intensity, the selected pulse duration, and the selected treatment session duration are sufficient to heat the target nerve to the temperature of 40 to 45 degrees C.

In Example 37, the subject matter of any one or more of Examples 33-36 optionally include wherein applying the focalized energy to the target tissue further comprises incrementally heating the target tissue to the temperature.

In Example 38, the subject matter of any one or more of Examples 33-37 optionally include calibrating the signal based on activation of pain receptors or a referred sensation reported by the subject.

In Example 39, the subject matter of any one or more of Examples 33-38 optionally include wherein the stimulation transducer comprises an ultrasound transducer and the stimulation controller comprises an ultrasound controller, and wherein generating the focalized energy further comprises generating an ultrasound frequency from 2 to 10 MHz.

In Example 39, the subject matter of Example 38 optionally includes wherein generating the signal indicating the selected pulse intensity, the selected pulse duration, and the selected treatment session duration further comprises generating the signal wherein the pulse duration is 3 seconds per minute and the selected treatment session duration is 30 minutes.

In Example 40, the subject matter of any one or more of Examples 33-39 optionally include wherein generating the signal indicating the selected pulse intensity, the selected pulse duration, and the selected treatment session duration further comprises generating the signal wherein the pulse duration is 3 seconds per minute and the selected treatment session duration is 30 minutes.

In Example 41, the subject matter of any one or more of Examples 33-40 optionally include wherein the selected pulse intensity is from 10 to 200 W/cm².

In Example 42, the subject matter of any one or more of Examples 33-41 optionally include wherein the selected pulse duration is from 1 second to 10 minutes.

In Example 43, the subject matter of any one or more of Examples 33-42 optionally include wherein applying the focalized energy to the target tissue at the selected depth below the surface of the skin of the subject further comprises applying the stimulation transducer to a contact area of skin comprising a surface area of 2 cm² to 20 cm².

In Example 44, the subject matter of any one or more of Examples 33-43 optionally include further comprising determining the selected depth from an adjustable depth range of the stimulation transducer from 0.1 cm to 30 cm prior to applying the focalized energy to the target tissue at the selected depth below the surface of the skin of the subject.

In Example 45, the subject matter of any one or more of Examples 33-44 optionally include wherein the target nerve comprises a vagus nerve.

In Example 46, the subject matter of any one or more of Examples 33-45 optionally include wherein the target nerve comprises Aδ or C fibers.

In Example 47, the subject matter of any one or more of Examples 33-46 optionally include wherein the thermo-sensitive ion channels comprise one or more of TRPV1, TRPV2, TRPV3, TRPM2, and TRPM3.

Each of these non-limiting embodiments and examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device, comprising:
a stimulation transducer that focally delivers energy to a target tissue at a selected depth below the surface of the skin of a subject, the target tissue comprising a target nerve comprising afferent nerve fibers with thermo-sensitive ion channels; and
a stimulation controller operably coupled to the stimulation transducer, wherein the stimulation controller generates a signal indicating a selected pulse intensity, a selected pulse duration, and a selected treatment session duration sufficient to heat the target tissue to a temperature from 40 to 45 degrees Celsius via application of energy by the stimulation transducer;
wherein, when the stimulation transducer receives the signal from the stimulation controller, the stimulation transducer focally delivers energy having the selected pulse intensity, the selected pulse duration, and the selected treatment session duration to heat the target tissue to reversibly activate the afferent nerve fibers with thermo-sensitive ion channels to send sensory information to the central nervous system without causing any permanent changes to the target tissue.

2. The device of claim 1, wherein the stimulation transducer comprises an ultrasound transducer and wherein the stimulation controller comprises an ultrasound controller.

3. The device of claim 2, wherein a distal end of the stimulation transducer includes a transduction medium interface.

4. The device of claim 2, wherein the stimulation transducer generates ultrasound frequencies from 2 to 10 MHz.

5. The device of claim 2, wherein the selected pulse intensity is from 10 to 200 W/cm².

6. The device of claim 2, wherein the selected pulse duration is from 1 second to 10 minutes.

7. The device of claim 6, wherein the selected pulse duration is from 3 seconds to 5 minutes.

8. The device of claim 7, wherein the selected pulse duration is from 3 seconds to 30 seconds.

9. The device of claim 2, wherein the selected treatment session duration is from 1 second to 24 hours.

10. The device of claim 9, wherein the selected treatment session duration is from 1 minute to 30 minutes.

11. The device of claim 1, wherein the stimulation transducer comprises at least one stimulation element.

12. The device of claim 1, wherein the stimulation transducer delivers energy focally across an adjustable depth range, the selected depth being selected from the adjustable depth range.

13. The device of claim 1, further comprising an imaging transducer and an imaging controller operably coupled to the imaging transducer.

14. The device of claim 1, wherein the stimulation transducer selectively activates the afferent nerve fibers with thermo-sensitive ion channels.

15. The device of claim 1, wherein the stimulation transducer does not cause any long-lasting changes to the target tissue.

16. A method, comprising:
generating a signal indicating a selected pulse intensity, a selected pulse duration, and a selected treatment session duration sufficient to heat a target tissue to a temperature from 40 to 45 degrees C. via a stimulation controller, the target tissue of a subject comprising a target nerve comprising afferent nerve fibers with thermo-sensitive ion channels;
transmitting the signal to a stimulation transducer;
generating focalized energy with the selected pulse intensity, the selected pulse duration, and the selected treatment session duration;
applying the focalized energy to the target tissue at a selected depth below the surface of the skin of a subject;
heating the target tissue to selectively and reversibly activate the afferent nerve fibers with thermo-sensitive ion channels to send selectively sensory information to a central nervous system of the subject without causing any long-lasting or permanent changes to the target tissue.

17. The method of claim 16, further comprising imaging the subject to locate the target nerve.

18. The method of claim 17, further comprising calibrating the signal based on a depth of the nerve from the skin surface and local tissue characteristics to ensure the selected depth, the selected pulse intensity, the selected pulse duration, and the selected treatment session duration are sufficient to heat the target nerve to the temperature from 40 to 45 degrees C.

19. The method of claim 16, wherein applying the focalized energy to the target tissue further comprises incrementally heating the target tissue to the temperature.

20. The method of claim 16, wherein the stimulation transducer comprises an ultrasound transducer and the stimulation controller comprises an ultrasound controller, and wherein generating the focalized energy further comprises generating an ultrasound frequency from 2 to 10 MHz.
